(19) United States
(12) Patent Application Publication
(10) Pub. No.: US 2026/0234504 A1
(43) Pub. Date: Aug. 13, 2026

(54) PROCESS FOR SEPARATING AND EXTRACTING NERVONIC ACID FROM XANTHOCERAS SORBIFOLIUM BUNGE

(71) Applicant: Nanchang University, Nanchang (CN)

(72) Inventors: Yan CHEN, Nanchang (CN); Hongbing CHEN, Nanchang (CN); Yong WU, Nanchang (CN); Jinyan GAO, Nanchang (CN); Xin LI, Nanchang (CN); Ping TONG, Nanchang (CN); Xuanyi MENG, Nanchang (CN); Shuangyan ZHENG, Nanchang (CN); Yanhai XIE, Nanchang (CN); Juanli YUAN, Nanchang (CN); Junhao FU, Nanchang (CN); Yuze TAO, Nanchang (CN)

(73) Assignee: Nanchang University, Nanchang (CN)

(21) Appl. No.: 19/533,086

(22) Filed: Feb. 6, 2026

(30) Foreign Application Priority Data
Feb. 12, 2025 (CN) 202510153517.2

(51) Int. Cl. C11C 1/08; B01D 67/00; B01D 71/68

(52) U.S. Cl. CPC C11C 1/08; B01D 67/00931; B01D 71/68

(57) ABSTRACT

The present disclosure relates to the technical field of natural product extraction, and particularly to a process for separating and extracting a nervonic acid from Xanthoceras sorbifolium Bunge. A preparation method thereof includes the following steps: extracting an Xanthoceras sorbifolium Bunge seed kernel to prepare Xanthoceras sorbifolium Bunge oil; refining the Xanthoceras sorbifolium Bunge oil by filtration through a composite polyethersulfone membrane and preparing a mixed fatty acid; and preparing a nervonic acid from the mixed fatty acid. In the present disclosure, molybdenum disulfide and hydrochloric acid-treated lithium fluoride are dispersed together in a solution. A composite polyethersulfone membrane obtained after treating a polyethersulfone membrane with a composite nanomaterial prepared by treatment with molybdenum disulfide and lithium fluoride, does not produce contamination and has an adsorption effect on an impurity in the Xanthoceras sorbifolium Bunge oil.

### RELATED APPLICATIONS

The present patent document claims the benefit of priority to patent application No. 202510153517.2, filed Feb. 12, 2025, and entitled “PROCESS FOR SEPARATING AND EXTRACTING NERVONIC ACID FROM XANTHOCERAS SORBIFOLIUM BUNGE,” the entire contents of each of which are incorporated herein by reference.

### BACKGROUND

### 1. Technical Field

The present disclosure relates to the technical field of natural product extraction, and particularly to a process for separating and extracting a nervonic acid from Xanthoceras sorbifolium Bunge.

### 2. Background Information

A nervonic acid is a long-chain saturated fatty acid which exists widely in nature, especially of a high content in certain plant and animal tissues. The nervonic acid plays an important physiological role in an organism, especially in a nervous system. It is one of the main ingredients of a myelin lipid and plays a key role in maintaining the structure and function of a nerve cell. Therefore, the nervonic acid has broad application prospects in the fields of medicine, health-care food and the like.

The nervonic acid is mainly derived from certain plant oil, for example palm oil, perilla oil, and acer truncatum oil, and the like oil extracted from plants. Among these, Xanthoceras sorbifolium Bunge oil is plant oil containing a relatively high proportion of the nervonic acid. The Xanthoceras sorbifolium Bunge oil is mainly extracted from a Xanthoceras sorbifolium Bunge seed. The Xanthoceras sorbifolium Bunge seed is rich in oil, and the Xanthoceras sorbifolium Bunge oil is typically prepared by pressing or solvent extraction. However, the Xanthoceras sorbifolium Bunge oil prepared by extraction further requires degumming, purification and the like steps to reduce impurities in the Xanthoceras sorbifolium Bunge oil. General vegetable oil degumming includes hydration degumming and acidification degumming, both of which belong to chemical degumming, require precise control of reaction times and conditions, and have complex operation steps. Performing physical filtration with a membrane is also a method for reducing impurities in oil. However, a conventional nanofiltration membrane, such as a polyethersulfone membrane, has insufficient hydrophilicity during filtration, and has low filtration efficiency and a small filtration effect on impurities in the Xanthoceras sorbifolium Bunge oil, and the biocompatibility of the polyethersulfone membrane is not high. Therefore, the conventional polyethersulfone membrane is generally not used for refining the Xanthoceras sorbifolium Bunge oil. Therefore, the present disclosure provides a process for separating and extracting a nervonic acid from Xanthoceras sorbifolium Bunge. After the Xanthoceras sorbifolium Bunge oil is extracted from the Xanthoceras sorbifolium Bunge, the Xanthoceras sorbifolium Bunge oil is subjected to membrane filtration through a prepared composite polyethersulfone membrane to complete degumming, purification, and refining steps. The biocompatibility of the composite polyethersulfone membrane is high and can improve the extraction rate and purity of the nervonic acid extracted from the Xanthoceras sorbifolium Bunge oil.

### BRIEF SUMMARY

In view of the shortcomings of the prior art, an objective of the present disclosure is to provide a process for separating and extracting a nervonic acid from Xanthoceras sorbifolium Bunge.

A process for separating and extracting a nervonic acid from Xanthoceras sorbifolium Bunge includes the following steps:
S1: preparation of Xanthoceras sorbifolium Bunge oil by extraction from a Xanthoceras sorbifolium Bunge seed kernel
crushing and then sieving a dried Xanthoceras sorbifolium Bunge seed, putting into an extraction kettle, releasing product oil from a separation tank of the extraction kettle after extraction, then centrifuging the product oil, filtering, and drying to obtain Xanthoceras sorbifolium Bunge oil;
S2: refining of the Xanthoceras sorbifolium Bunge oil by filtration through a composite polyethersulfone membrane and preparation of a mixed fatty acid
setting an organic membrane in an organic membrane separation device as a composite polyethersulfone membrane, wherein the composite polyethersulfone membrane is a polyethersulfone membrane loaded with a composite nanomaterial, and the composite nanomaterial is prepared from two-dimensional molybdenum disulfide powder as a raw material; adding the Xanthoceras sorbifolium Bunge oil into a separation tank of the organic membrane separation device and filtering through the composite polyethersulfone membrane to obtain refined Xanthoceras sorbifolium Bunge oil; weighing and adding the refined Xanthoceras sorbifolium Bunge oil into a NaOH-ethanol mixed solution, heating to obtain saponified oil, adding an equal volume of deionized water into the saponified oil for dilution, and extracting with n-hexane to obtain a mixed fatty acid;
S3: preparation of a nervonic acid from the mixed fatty acid
mixing the mixed fatty acid with an ethanol solution, freezing to crystallize, then performing suction filtration with a vacuum circulating water pump, and drying a filter cake to obtain a nervonic acid.

Further, the preparation of by extraction from a Xanthoceras sorbifolium Bunge in the step S1 includes the following steps:
S1.1: crushing and then sieving a dried Xanthoceras sorbifolium Bunge seed through an 80-mesh sieve, putting into an extraction kettle, sealing, vacuuming to a gauge pressure of −0.6 MPa, and then introducing tetrafluoroethane as an extraction solvent from a solvent tank into the extraction kettle until a liquid level of the extraction solvent submerges the Xanthoceras sorbifolium Bunge seed raw material as observed through sight glass;
S1.2: heating to 40-50° C. under a stirring state, and always maintaining a pressure of the extraction kettle at a gauge pressure of 1.0 MPa to fully dissolve oil in the Xanthoceras sorbifolium Bunge seed, wherein an extraction time is 50-60 min; after extraction, introducing an extract liquor into a separation tank with a pressurization pump, evaporating by heating to remove the extraction solvent, and recovering the evaporated extraction solvent vapor into a solvent tank for reuse by a system compressor through a condenser, wherein the product oil is in the separation tank and is released from the separation tank; and
S1.3: then centrifugally separating the product oil at a rotation speed of 8,000-8,200 r/min for 5-10 min, filtering, and drying at 105-110° C. for 3-3.5 h to obtain Xanthoceras sorbifolium Bunge oil.

Further, the refining of the by filtration through a composite polyethersulfone membrane and preparation of a in the step S2 includes the following steps:
S2.1: replacing the organic membrane in the organic membrane separation device with the composite polyethersulfone membrane, wherein the composite polyethersulfone membrane is a polyethersulfone membrane loaded with a composite nanomaterial, and the composite nanomaterial is prepared from two-dimensional molybdenum disulfide powder as a raw material, adding the Xanthoceras sorbifolium Bunge oil into the separation tank of the organic membrane separation device, starting the pump to a rotation speed of 40 Hz, adjusting a pressure regulating valve of a feed liquid return pipe to set a pressure to 1.9 MPa, and filtering the Xanthoceras sorbifolium Bunge oil through the composite polyethersulfone membrane to obtain refined Xanthoceras sorbifolium Bunge oil;
S2.2: weighing 10-12 parts by mass of the refined Xanthoceras sorbifolium Bunge oil, adding 50-60 parts by mass of a NaOH-ethanol mixed solution, maintaining the temperature at 65-70° C. through an oil bath pot, setting a heating time to 1.5-2 h, and after end of the heating, cooling to room temperature of 24-26° C. to obtain saponified oil; and
S2.3: adding an equal volume of deionized water into the saponified oil for dilution, then adding 3 mol/L of hydrochloric acid to adjust a pH value of the system to 6-6.5, extracting with n-hexane, repeating the operation twice, collecting the n-hexane solutions from the two extractions, placing the n-hexane solutions into a rotary evaporator for concentration, and spinning the n-hexane to dryness to obtain a mixed fatty acid.

Further, a method for preparing the two-dimensional molybdenum disulfide powder includes the following steps:
S2.1.1: adding lithium fluoride into 3 mol/L of hydrochloric acid at a solid-to-liquid ratio of 1 g:(400-500) mL, magnetically stirring for 25 min, then adding molybdenum disulfide with the same mass as the lithium fluoride, heating to 45-50° C., magnetically stirring for 5-6 h, and centrifuging and taking a precipitate to obtain a solid precipitate; and
S2.1.2: washing the solid precipitate with a LiCl solution for 5-6 times, then adding deionized water at a solid-to-liquid ratio of 1 g:(250-300) mL, subjecting to ultrasonic treatment for 30-50 min, centrifuging, taking the supernatant, and freeze-drying the supernatant at minus 20-minus 15° C. for 48-52 h to obtain two-dimensional molybdenum disulfide powder.

Further, a method for preparing the composite nanomaterial includes the following steps:
S2.1.3: taking and adding the two-dimensional molybdenum disulfide powder prepared in the step S2.1.2 into deionized water at a solid-to-liquid ratio of 3 g:(100-200) mL, subjecting to ultrasonic treatment for 20-30 min, and magnetically stirring for 40-45 min to obtain a molybdenum disulfide dispersion; and
S2.1.4: then adding a phytic acid solution at 15-20% of a total volume of the system, subjecting to ultrasonic treatment for 30-35 min to obtain a dispersion, heating the dispersion to 180-185° C., reacting for 12-13 h, then allowing to stand and cool to room temperature of 24-26° C., centrifuging a cooled product, removing the supernatant, and freeze-drying at 0° C. for 24-25 h to obtain a composite nanomaterial.

Further, a method for preparing the composite polyethersulfone membrane comprises the following steps:
S2.1.5: taking and adding 5-10 parts by mass of piperazine into 100-150 parts by mass of deionized water, then adding 10-20 parts by mass of the composite nanomaterial prepared in the step S2.1.4, and stirring for 20-30 min to obtain a suspension;
S2.1.6: adding the suspension dropwise onto a surface of a polyethersulfone-based membrane to fully infiltrate the surface, and oven-drying under an environment of 60-65° C. after 3-5 min to obtain a primarily-treated polyethersulfone-based membrane; and
S2.1.7: adding an TMC organic phase solution dropwise onto a surface of the primarily-treated polyethersulfone-based membrane to fully infiltrate the surface, then allowing to stand for 30-50 s, and thermally crosslinking in an oven at 90-100° C. for 30-40 s to obtain a composite polyethersulfone membrane.

Further, the preparation of a nervonic acid from the mixed fatty acid in the step S3 includes the following steps:
S3.1: mixing the mixed fatty acid with an ethanol solution of a concentration of 90% at a solid-to-liquid ratio of 1 g:(3-4) mL, and placing in an environment of −20° C. for freezing and crystallizing for 3-4 h; and
S3.2: then performing suction filtration with a vacuum circulating water pump, ending the suction filtration when a filter cake does not contain the ethanol solution, and drying the filter cake at 20-25° C. for 24-30 h to obtain a nervonic acid.

Further, a concentration of the phytic acid solution in the step S2.1.4 is 0.2-0.8 wt %.

Further, the TMC organic phase solution in the step S2.1.7 is specifically a solution with a mass fraction of 2-3% formulated by dissolving trimesoyl chloride in n-hexane.

Further, a volume ratio of NaOH to ethanol in the NaOH-ethanol mixed solution in the step S2.2 is 2:(3-4).

Compared with the prior art, the present disclosure has at least the following beneficial effects:
1. In the present disclosure, molybdenum disulfide and hydrochloric acid-treated lithium fluoride are dispersed together in a solution, the lithium fluoride can conduct surface treatment on the molybdenum disulfide, and the introduction of the lithium fluoride can help the molybdenum disulfide to separate into a two-dimensional structure with a single layer or few layers by a physical stripping method, thereby obtaining a high specific surface area and excellent physicochemical properties. Furthermore, through the treatment with the lithium fluoride, the ions of the lithium fluoride interact with a surface of the molybdenum disulfide to form stable chemical bonds, improving the stability of the molybdenum disulfide and allowing the two-dimensional structure to be better dispersed in the solvent to form a stable colloidal dispersion. Both the molybdenum disulfide and the lithium fluoride are environmentally friendly. A composite polyethersulfone membrane obtained after treating a polyethersulfone membrane with a composite nanomaterial prepared by treatment with molybdenum disulfide and lithium fluoride, does not produce contamination and has an adsorption effect on an impurity in the Xanthoceras sorbifolium Bunge oil. During the filtration of the Xanthoceras sorbifolium Bunge oil through the polyethersulfone membrane, other impurities are adsorbed, thereby refining the Xanthoceras sorbifolium Bunge oil, reducing the content of extraction solvents and other impurities in the Xanthoceras sorbifolium Bunge oil, and improving the extraction rate and purity of the nervonic acid from the Xanthoceras sorbifolium Bunge oil.
2. In the present disclosure, the surface of the two-dimensional molybdenum disulfide powder is modified with the phytic acid. The phosphate groups in the phytic acid can form stable chemical bonds on the surface of two-dimensional molybdenum disulfide, and improve the chemical stability of the two-dimensional molybdenum disulfide, thereby reducing the oxidation of the molybdenum disulfide in the air. This extends the stability and service life of the polyethersulfone membrane of which the adsorption performance is improved by the introduction of the molybdenum disulfide. Furthermore, the nanomaterial prepared from the molybdenum disulfide treated with the phytic acid has a large specific surface area and improved surface activity, has better selective adsorption and catalytic activity. The high catalytic activity can improve the subsequent adhesion on the polyethersulfone membrane, and the high adsorption can give the composite polyethersulfone membrane better filtration and impurity adsorption capabilities, thereby improving the purity of the nervonic acid extracted from the Xanthoceras sorbifolium Bunge oil.
3. In the present disclosure, the Xanthoceras sorbifolium Bunge oil is refined through the composite polyethersulfone membrane. After the Xanthoceras sorbifolium Bunge oil is extracted from the Xanthoceras sorbifolium Bunge seed kernel, the oil still needs to undergo degumming, purification and the like steps. The composite polyethersulfone membrane can directly conduct degumming and purification on the Xanthoceras sorbifolium Bunge oil through physical filtration. Furthermore, during the degumming process, the composite nanomaterial using the molybdenum disulfide as a raw material loaded on the composite polyethersulfone membrane will also adsorb impurities in the Xanthoceras sorbifolium Bunge oil, thereby improving the purity of the Xanthoceras sorbifolium Bunge oil and further improving the extraction rate and purity of the nervonic acid extracted from the Xanthoceras sorbifolium Bunge oil.

### BRIEF DESCRIPTION OF THE DRAWINGS

The accompanying drawings, which are incorporated herein and constitute a part of the specification, illustrate embodiments of the present disclosure, and, together with the specification, further serve to explain the principles of the present disclosure and enable those skilled in the relevant art to implement and use the present disclosure.

FIG. 1 is a flow diagram of a process for separating and extracting a nervonic acid from Xanthoceras sorbifolium Bunge.

### DETAILED DESCRIPTION OF THE DRAWINGS AND THE PRESENTLY PREFERRED EMBODIMENTS

A process for separating and extracting a nervonic acid from Xanthoceras sorbifolium Bunge provided by the present invention will be described in detail hereafter with reference to the accompanying drawings and specific embodiments. Meanwhile, it should be illustrated here that, in order to make the embodiments more detailed, the following examples are the best and preferred embodiments. For some well-known technologies, those skilled in the art can also use other alternative manners to implement them. Moreover, the accompanying drawings are only for more specific description of the embodiments, and are not intended to specifically limit the present disclosure.

### Example 1

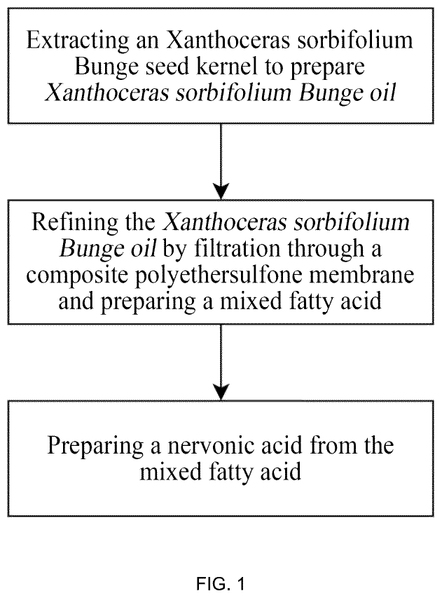

A process for separating and extracting a nervonic acid from Xanthoceras sorbifolium Bunge, as shown in FIG. 1, included the following steps:
S1: preparation of Xanthoceras sorbifolium Bunge oil by extraction from a Xanthoceras sorbifolium Bunge seed kernel
S1.1: a dried Xanthoceras sorbifolium Bunge seed was crushed and then sieved through an 80-mesh sieve, put into an extraction kettle, sealed, and vacuumed to a gauge pressure of −0.6 MPa, and then tetrafluoroethane was introduced as an extraction solvent from a solvent tank into the extraction kettle until a liquid level of the extraction solvent submerged the Xanthoceras sorbifolium Bunge seed raw material as observed through sight glass;
S1.2: heating was performed to 40° C. under a stirring state, and a pressure of the extraction kettle was always maintained at a gauge pressure of 1.0 MPa to fully dissolve oil in the Xanthoceras sorbifolium Bunge seed, where an extraction time was 50 min; after extraction, an extract liquor was introduced into a separation tank with a pressurization pump, evaporating was conducted by heating to remove the extraction solvent, and the evaporated extraction solvent vapor was recovered into a solvent tank for reuse by a system compressor through a condenser, where the product oil was in the separation tank and was released from the separation tank; and
S1.3: then the product oil was centrifugally separated at a rotation speed of 8,000 r/min for 5 min, filtered, and dried at 105° C. for 3 h to obtain Xanthoceras sorbifolium Bunge oil.
S2: refining of the Xanthoceras sorbifolium Bunge oil by filtration through a composite polyethersulfone membrane and preparation of a mixed fatty acid
S2.1: the organic membrane in the organic membrane separation device was replaced with the composite polyethersulfone membrane, where the composite polyethersulfone membrane was a polyethersulfone membrane loaded with a composite nanomaterial, and the composite nanomaterial was prepared from two-dimensional molybdenum disulfide powder as a raw material, the Xanthoceras sorbifolium Bunge oil was added into the separation tank of the organic membrane separation device, the pump was started to a rotation speed of 40 Hz, a pressure regulating valve of a feed liquid return pipe was adjusted to set a pressure to 1.9 MPa, and the Xanthoceras sorbifolium Bunge oil was filtered through the composite polyethersulfone membrane to obtain refined Xanthoceras sorbifolium Bunge oil;
where a method for preparing the two-dimensional molybdenum disulfide powder was specifically:
S2.1.1: lithium fluoride was added into 3 mol/L of hydrochloric acid at a solid-to-liquid ratio of 1 g:400 mL, magnetically stirred for 25 min, then added with molybdenum disulfide with the same mass as the lithium fluoride, heated to 45° C., magnetically stirred for 5 h, and centrifuged, and a precipitate was taken to obtain a solid precipitate; and
S2.1.2: the solid precipitate was washed with a LiCl solution for 5 times, then added with deionized water at a solid-to-liquid ratio of 1 g:250 mL, subjected to ultrasonic treatment for 30 min, and centrifuged, and the supernatant was taken, and freeze-dried at minus 20° C. for 48 h to obtain two-dimensional molybdenum disulfide powder.

A method for preparing the composite nanomaterial was specifically:
S2.1.3: the two-dimensional molybdenum disulfide powder prepared in the step S2.1.2 was taken and added into deionized water at a solid-to-liquid ratio of 3 g: 100 mL, subjected to ultrasonic treatment for 20 min, and magnetically stirred for 40 min to obtain a molybdenum disulfide dispersion; and
S2.1.4: then a phytic acid solution of a concentration of 0.2 wt % was added at 15% of a total volume of the system, and subjected to ultrasonic treatment for 30 min to obtain a dispersion, the dispersion was heated to 180° C., reacted for 12 h, and then allowed to stand and cool to room temperature of 24° C., a cooled product was centrifuged, the supernatant was removed, and freeze-drying was conducted at 0° C. for 24 h to obtain a composite nanomaterial.

A method for preparing the composite polyethersulfone membrane was specifically:
S2.1.5:5 parts by mass of piperazine was taken and added into 100 parts by mass of deionized water, then added with 10 parts by mass of the composite nanomaterial prepared in the step S2.1.4, and stirred for 20 min to obtain a suspension;
S2.1.6: the suspension was added dropwise onto a surface of a polyethersulfone-based membrane to fully infiltrate the surface, and oven-dried under an environment of 60° C. after 3 min to obtain a primarily-treated polyethersulfone-based membrane; and
S2.1.7: an TMC organic phase solution was added dropwise onto a surface of the primarily-treated polyethersulfone-based membrane to fully infiltrate the surface, where the TMC organic phase solution was specifically a solution with a mass fraction of 2% formulated by dissolving trimesoyl chloride in n-hexane, and then the infiltrated surface was allowed to stand for 30 s, and thermally crosslinked in an oven at 90° C. for 30 s to obtain a composite polyethersulfone membrane.
S2.2: 10 parts by mass of the refined Xanthoceras sorbifolium Bunge oil was weighed, and added with 50 parts by mass of a NaOH-ethanol mixed solution, where a volume ratio of NaOH to ethanol in the NaOH-ethanol mixed solution was 2:3, the temperature was maintained at 65° C. through an oil bath pot, a heating time was set to 1.5 h, and after end of the heating, cooling was conducted to room temperature of 24° C. to obtain saponified oil; and
S2.3: an equal volume of deionized water was added into the saponified oil for dilution, then added with 3 mol/L of hydrochloric acid to adjust a pH value of the system to 6, and extracted with n-hexane, the operation was repeated twice, and the n-hexane solutions from the two extractions was collected, and placed into a rotary evaporator for concentration, and the n-hexane was spun to dryness to obtain a mixed fatty acid.
S3: preparation of a nervonic acid from the mixed fatty acid
S3.1: the mixed fatty acid was mixed with an ethanol solution of a concentration of 90% at a solid-to-liquid ratio of 1 g:3 mL, and placed in an environment of −20° C. for freezing and crystallizing for 3 h; and
S3.2: then suction filtration was performed with a vacuum circulating water pump, the suction filtration was ended when a filter cake did not contain the ethanol solution, and the filter cake was dried at 20° C. for 24 h to obtain a nervonic acid.

### Example 2

A process for separating and extracting a nervonic acid from Xanthoceras sorbifolium Bunge, as shown in FIG. 1, included the following steps:
S1: preparation of Xanthoceras sorbifolium Bunge oil by extraction from a Xanthoceras sorbifolium Bunge seed kernel
S1.1: a dried Xanthoceras sorbifolium Bunge seed was crushed and then sieved through an 80-mesh sieve, put into an extraction kettle, sealed, and vacuumed to a gauge pressure of −0.6 MPa, and then tetrafluoroethane was introduced as an extraction solvent from a solvent tank into the extraction kettle until a liquid level of the extraction solvent submerged the Xanthoceras sorbifolium Bunge seed raw material as observed through sight glass;
S1.2: heating was performed to 50° C. under a stirring state, and a pressure of the extraction kettle was always maintained at a gauge pressure of 1.0 MPa to fully dissolve oil in the Xanthoceras sorbifolium Bunge seed, where an extraction time was 60 min; after extraction, an extract liquor was introduced into a separation tank with a pressurization pump, evaporating was conducted by heating to remove the extraction solvent, and the evaporated extraction solvent vapor was recovered into a solvent tank for reuse by a system compressor through a condenser, where the product oil was in the separation tank and was released from the separation tank; and
S1.3: then the product oil was centrifugally separated at a rotation speed of 8,200 r/min for 10 min, filtered, and dried at 110° C. for 3.5 h to obtain Xanthoceras sorbifolium Bunge oil.
S2: refining of the Xanthoceras sorbifolium Bunge oil by filtration through a composite polyethersulfone membrane and preparation of a mixed fatty acid
S2.1: the organic membrane in the organic membrane separation device was replaced with the composite polyethersulfone membrane, where the composite polyethersulfone membrane was a polyethersulfone membrane loaded with a composite nanomaterial, and the composite nanomaterial was prepared from two-dimensional molybdenum disulfide powder as a raw material, the Xanthoceras sorbifolium Bunge oil was added into the separation tank of the organic membrane separation device, the pump was started to a rotation speed of 40 Hz, a pressure regulating valve of a feed liquid return pipe was adjusted to set a pressure to 1.9 MPa, and the Xanthoceras sorbifolium Bunge oil was filtered through the composite polyethersulfone membrane to obtain refined Xanthoceras sorbifolium Bunge oil;
where a method for preparing the two-dimensional molybdenum disulfide powder was specifically:
S2.1.1: lithium fluoride was added into 3 mol/L of hydrochloric acid at a solid-to-liquid ratio of 1 g:400 mL, magnetically stirred for 25 min, then added with molybdenum disulfide with the same mass as the lithium fluoride, heated to 50° C., magnetically stirred for 6 h, and centrifuged, and a precipitate was taken to obtain a solid precipitate; and
S2.1.2: the solid precipitate was washed with a LiCl solution for 6 times, then added with deionized water at a solid-to-liquid ratio of 1 g:250 mL, subjected to ultrasonic treatment for 50 min, and centrifuged, and the supernatant was taken, and freeze-dried at minus 15° C. for 52 h to obtain two-dimensional molybdenum disulfide powder.

A method for preparing the composite nanomaterial was specifically:
S2.1.3: the two-dimensional molybdenum disulfide powder was taken and added into deionized water at a solid-to-liquid ratio of 3 g:100 mL, subjected to ultrasonic treatment for 30 min, and magnetically stirred for 45 min to obtain a molybdenum disulfide dispersion; and
S2.1.4: then a phytic acid solution of a concentration of 0.8 wt % was added at 15% of a total volume of the system, and subjected to ultrasonic treatment for 20 min to obtain a dispersion, the dispersion was heated to 185° C., reacted for 13 h, and then allowed to stand and cool to room temperature of 26° C., a cooled product was centrifuged, the supernatant was removed, and freeze-drying was conducted at 0° C. for 25 h to obtain a composite nanomaterial.

A method for preparing the composite polyethersulfone membrane was specifically:
S2.1.5:5 parts by mass of piperazine was taken and added into 100 parts by mass of deionized water, then added with 10 parts by mass of the composite nanomaterial, and stirred for 30 min to obtain a suspension;
S2.1.6: the suspension was added dropwise onto a surface of a polyethersulfone-based membrane to fully infiltrate the surface, and oven-dried under an environment of 65° C. after 5 min to obtain a primarily-treated polyethersulfone-based membrane; and
S2.1.7: an TMC organic phase solution was added dropwise onto a surface of the primarily-treated polyethersulfone-based membrane to fully infiltrate the surface, where the TMC organic phase solution was specifically a solution with a mass fraction of 2% formulated by dissolving trimesoyl chloride in n-hexane, and then the infiltrated surface was allowed to stand for 50 s, and thermally crosslinked in an oven at 100° C. for 40 s to obtain a composite polyethersulfone membrane.
S2.2: 10 parts by mass of the refined Xanthoceras sorbifolium Bunge oil was weighed, and added with 50 parts by mass of a NaOH-ethanol mixed solution, where a volume ratio of NaOH to ethanol in the NaOH-ethanol mixed solution was 2:3, the temperature was maintained at 70° C. through an oil bath pot, a heating time was set to 2 h, and after end of the heating, cooling was conducted to room temperature of 26° C. to obtain saponified oil; and
S2.3: an equal volume of deionized water was added into the saponified oil for dilution, then added with 3 mol/L of hydrochloric acid to adjust a pH value of the system to 6.5, and extracted with n-hexane, the operation was repeated twice, and the n-hexane solutions from the two extractions was collected, and placed into a rotary evaporator for concentration, and the n-hexane was spun to dryness to obtain a mixed fatty acid.
S3: preparation of a nervonic acid from the mixed fatty acid
S3.1: the mixed fatty acid was mixed with an ethanol solution of a concentration of 90% at a solid-to-liquid ratio of 1 g:3 mL, and placed in an environment of −20° C. for freezing and crystallizing for 4 h; and
S3.2: then suction filtration was performed with a vacuum circulating water pump, the suction filtration was ended when a filter cake did not contain the ethanol solution, and the filter cake was dried at 25° C. for 30 h to obtain a nervonic acid.

### Example 3

A process for separating and extracting a nervonic acid from Xanthoceras sorbifolium Bunge, as shown in FIG. 1, included the following steps:
S1: preparation of Xanthoceras sorbifolium Bunge oil by extraction from a Xanthoceras sorbifolium Bunge seed kernel
S1.1: a dried Xanthoceras sorbifolium Bunge seed was crushed and then sieved through an 80-mesh sieve, put into an extraction kettle, sealed, and vacuumed to a gauge pressure of −0.6 MPa, and then tetrafluoroethane was introduced as an extraction solvent from a solvent tank into the extraction kettle until a liquid level of the extraction solvent submerged the Xanthoceras sorbifolium Bunge seed raw material as observed through sight glass;
S1.2: heating was performed to 40° C. under a stirring state, and a pressure of the extraction kettle was always maintained at a gauge pressure of 1.0 MPa to fully dissolve oil in the Xanthoceras sorbifolium Bunge seed, where an extraction time was 50 min; after extraction, an extract liquor was introduced into a separation tank with a pressurization pump, evaporating was conducted by heating to remove the extraction solvent, and the evaporated extraction solvent vapor was recovered into a solvent tank for reuse by a system compressor through a condenser, where the product oil was in the separation tank and was released from the separation tank; and
S1.3: then the product oil was centrifugally separated at a rotation speed of 8,000 r/min for 5 min, filtered, and dried at 105° C. for 3 h to obtain Xanthoceras sorbifolium Bunge oil.
S2: refining of the Xanthoceras sorbifolium Bunge oil by filtration through a composite polyethersulfone membrane and preparation of a mixed fatty acid
S2.1: the organic membrane in the organic membrane separation device was replaced with the composite polyethersulfone membrane, where the composite polyethersulfone membrane was a polyethersulfone membrane loaded with a composite nanomaterial, and the composite nanomaterial was prepared from two-dimensional molybdenum disulfide powder as a raw material, the Xanthoceras sorbifolium Bunge oil was added into the separation tank of the organic membrane separation device, the pump was started to a rotation speed of 40 Hz, a pressure regulating valve of a feed liquid return pipe was adjusted to set a pressure to 1.9 MPa, and the Xanthoceras sorbifolium Bunge oil was filtered through the composite polyethersulfone membrane to obtain refined Xanthoceras sorbifolium Bunge oil;
where a method for preparing the two-dimensional molybdenum disulfide powder was specifically:
S2.1.1: lithium fluoride was added into 3 mol/L of hydrochloric acid at a solid-to-liquid ratio of 1 g:500 mL, magnetically stirred for 25 min, then added with molybdenum disulfide with the same mass as the lithium fluoride, heated to 45° C., magnetically stirred for 5 h, and centrifuged, and a precipitate was taken to obtain a solid precipitate; and
S2.1.2: the solid precipitate was washed with a LiCl solution for 5 times, then added with deionized water at a solid-to-liquid ratio of 1 g:300 mL, subjected to ultrasonic treatment for 30 min, and centrifuged, and the supernatant was taken, and freeze-dried at minus 20° C. for 48 h to obtain two-dimensional molybdenum disulfide powder.

A method for preparing the composite nanomaterial was specifically:
S2.1.3: the two-dimensional molybdenum disulfide powder was taken and added into deionized water at a solid-to-liquid ratio of 3 g:200 mL, subjected to ultrasonic treatment for 20 min, and magnetically stirred for 40 min to obtain a molybdenum disulfide dispersion; and
S2.1.4: then a phytic acid solution of a concentration of 0.2 wt % was added at 20% of a total volume of the system, and subjected to ultrasonic treatment for 30 min to obtain a dispersion, the dispersion was heated to 180° C., reacted for 12 h, and then allowed to stand and cool to room temperature of 24° C., a cooled product was centrifuged, the supernatant was removed, and freeze-drying was conducted at 0° C. for 24 h to obtain a composite nanomaterial.

A method for preparing the composite polyethersulfone membrane was specifically:
S2.1.5: 10 parts by mass of piperazine was taken and added into 150 parts by mass of deionized water, then added with 10 parts by mass of the composite nanomaterial, and stirred for 20 min to obtain a suspension;
S2.1.6: the suspension was added dropwise onto a surface of a polyethersulfone-based membrane to fully infiltrate the surface, and oven-dried under an environment of 60° C. after 3 min to obtain a primarily-treated polyethersulfone-based membrane; and
S2.1.7: an TMC organic phase solution was added dropwise onto a surface of the primarily-treated polyethersulfone-based membrane to fully infiltrate the surface, where the TMC organic phase solution was specifically a solution with a mass fraction of 3% formulated by dissolving trimesoyl chloride in n-hexane, and then the infiltrated surface was allowed to stand for 30 s, and thermally crosslinked in an oven at 90° C. for 30 s to obtain a composite polyethersulfone membrane.
S2.2: 12 parts by mass of the refined Xanthoceras sorbifolium Bunge oil was weighed, and added with 60 parts by mass of a NaOH-ethanol mixed solution, where a volume ratio of NaOH to ethanol in the NaOH-ethanol mixed solution was 2:4, the temperature was maintained at 65° C. through an oil bath pot, a heating time was set to 1.5 h, and after end of the heating, cooling was conducted to room temperature of 24° C. to obtain saponified oil; and
S2.3: an equal volume of deionized water was added into the saponified oil for dilution, then added with 3 mol/L of hydrochloric acid to adjust a pH value of the system to 6, and extracted with n-hexane, the operation was repeated twice, and the n-hexane solutions from the two extractions was collected, and placed into a rotary evaporator for concentration, and the n-hexane was spun to dryness to obtain a mixed fatty acid.
S3: preparation of a nervonic acid from the mixed fatty acid
S3.1: the mixed fatty acid was mixed with an ethanol solution of a concentration of 90% at a solid-to-liquid ratio of 1 g:4 mL, and placed in an environment of −20° C. for freezing and crystallizing for 3 h; and
S3.2: then suction filtration was performed with a vacuum circulating water pump, the suction filtration was ended when a filter cake did not contain the ethanol solution, and the filter cake was dried at 20° C. for 24 h to obtain a nervonic acid.

### Comparative Example 1

Compared with Example 1, Comparative Example 1 differed in that the step S2.1 was not conducted, and the mixed fatty acid was prepared directly from the Xanthoceras sorbifolium Bunge oil, which was specifically: “step 1: 10 parts by mass of the Xanthoceras sorbifolium Bunge oil was weighed, and added with 50 parts by mass of a NaOH-ethanol mixed solution, where a volume ratio of NaOH to ethanol in the NaOH-ethanol mixed solution was 2:3, the temperature was maintained at 65° C. through an oil bath pot, a heating time was set to 1.5 h, and after end of the heating, cooling was conducted to room temperature of 24° C. to obtain saponified oil; and
step 2: an equal volume of deionized water was added into the saponified oil for dilution, then added with 3 mol/L of hydrochloric acid to adjust a pH value of the system to 6, and extracted with n-hexane, the operation was repeated twice, and the n-hexane solutions from the two extractions was collected, and placed into a rotary evaporator for concentration, and the n-hexane was spun to dryness to obtain a mixed fatty acid”. The remaining steps remained unchanged, and the extraction rate and purity of the nervonic acid in Comparative Example 1 were calculated.

### Comparative Example 2

Compared with Example 1, Comparative Example 2 differed in that the steps S2.1.1 and S2.1.2 were not conducted, and in the step S2.1.3, molybdenum disulfide powder was used instead of the two-dimensional molybdenum disulfide powder, which was specifically: “S2.1.3: the molybdenum disulfide powder was taken and added into deionized water at a solid-to-liquid ratio of 3 g:100 mL, subjected to ultrasonic treatment for 20 min, and magnetically stirred for 40 min to obtain a molybdenum disulfide dispersion”. The remaining steps remained unchanged, and the extraction rate and purity of the nervonic acid in Comparative Example 2 were calculated.

### Comparative Example 3

Compared with Example 1, Comparative Example 3 differed in that the steps S2.1.3 and S2.1.4 were not conducted, and in the step S2.1.5, two-dimensional molybdenum disulfide powder was used instead of the composite nanomaterial, which was specifically: “S2.1.5:5 parts by mass of piperazine was taken and added into 100 parts by mass of deionized water, then added with 10 parts by mass of the molybdenum disulfide powder, and stirred for 20 min to obtain a suspension”. The remaining steps remained unchanged, and the extraction rate and purity of the nervonic acid in Comparative Example 3 were calculated.

### Comparative Example 4

Compared with Example 1, Comparative Example 4 differed in that in the step S2.1, the composite polyethersulfone membrane was not used, and a polyethersulfone membrane was used instead, which was specifically: “S2.1: the organic membrane in the organic membrane separation device was replaced with a commercially available polyethersulfone membrane, the Xanthoceras sorbifolium Bunge oil was added into the separation tank of the organic membrane separation device, the pump was started to a rotation speed of 40 Hz, a pressure regulating valve of a feed liquid return pipe was adjusted to set a pressure to 1.9 MPa, and the Xanthoceras sorbifolium Bunge oil was filtered through the composite polyethersulfone membrane to obtain refined Xanthoceras sorbifolium Bunge oil”. The remaining steps remained unchanged, and the extraction rate and purity of the nervonic acid in Comparative Example 4 were calculated.

the extraction rate of the nervonic acid=the mass of the nervonic acid/the mass of Xanthoceras sorbifolium Bunge oil*100%. Calculation of Extraction Rate of Nervonic Acid:

The purity of the nervonic acid was obtained by analysis via GC-MS.

The extraction rates and purities of the nervonic acid in Examples 1-3 were calculated, and summarized in Table 1 together with the data from Comparative Examples 1-4.

TABLE 1

| | Extraction | Purity |
|---|---|---|
| | rate (%) | (%) |
| Example 1 | 3.78 | 99.31 |
| Example 2 | 3.62 | 99.15 |
| Example 3 | 3.71 | 99.27 |
| Comparative | 2.66 | 96.28 |
| Example 1 | | |
| Comparative | 2.99 | 97.13 |
| Example 2 | | |
| Comparative | 2.85 | 97.69 |
| Example 3 | | |
| Comparative | 2.83 | 96.93 |
| Example 4 | | |

In Examples 1-3, the extraction rates were 3.78%, 3.62%, and 3.71%, respectively, and the purities of the nervonic acid were 99.31%, 99.15%, and 99.27%, respectively, which were all higher than those of the comparative examples. It could be seen that the process for separating and extracting a nervonic acid from Xanthoceras sorbifolium Bunge of the present invention has a higher extraction rate and purity.

In Comparative Example 1, the Xanthoceras sorbifolium Bunge oil was refined without use of the composite polyethersulfone membrane, the extraction rate of the nervonic acid was 2.66%, and the purity of the nervonic acid was 96.28%. It could be seen that without the use of the composite polyethersulfone membrane, impurities would still remain in the Xanthoceras sorbifolium Bunge oil, thereby affecting the purity and extraction rate of the nervonic acid.

In the Comparative Examples 2 and 3, the extraction rates were 2.99% and 2.85%, respectively, and the purities of the nervonic acid were 97.13% and 97.69%, respectively. It could be seen that the combination of lithium fluoride and molybdenum disulfide, as well as the surface modification of molybdenum disulfide with a phytic acid, both improved the adsorption capacity of the polyethersulfone membrane for impurities after the nanomaterial prepared with molybdenum disulfide as the main raw material was combined with the polyethersulfone membrane, thereby improving the purity and extraction rate of the nervonic acid.

In the Comparative Example 4, the extraction rate was 2.83%, and the purity was 96.93%. It could be seen that the polyethersulfone membrane had an influence on the improvement of the purity and extraction rate of the nervonic acid, while the composite polyethersulfone membrane prepared in the present disclosure had a greater improvement effect on the purity and extraction rate of the nervonic acid.

The aforementioned examples merely illustrate the principles and efficacy of the present disclosure, rather than limiting the present disclosure. Anyone skilled in the art can modify or change the aforementioned examples without departing from the spirit and scope of the present disclosure. Therefore, all equivalent modifications or changes made by those of ordinary skills in the art without departing from the spirit and technical idea disclosed in the present disclosure should still be covered by the claims of the present disclosure.

1. A process for separating and extracting a nervonic acid from Xanthoceras sorbifolium Bunge, comprising the following steps:
S1: preparation of Xanthoceras sorbifolium Bunge oil by extraction from a Xanthoceras sorbifolium Bunge seed kernel
crushing and then sieving a dried Xanthoceras sorbifolium Bunge seed, putting into an extraction kettle, releasing product oil from a separation tank of the extraction kettle after extraction, then centrifuging the product oil, filtering, and drying to obtain Xanthoceras sorbifolium Bunge oil;
S2: refining of the Xanthoceras sorbifolium Bunge oil by filtration through a composite polyethersulfone membrane and preparation of a mixed fatty acid
setting an organic membrane in an organic membrane separation device as a composite polyethersulfone membrane, wherein the composite polyethersulfone membrane is a polyethersulfone membrane loaded with a composite nanomaterial, and the composite nanomaterial is prepared from two-dimensional molybdenum disulfide powder as a raw material; adding the Xanthoceras sorbifolium Bunge oil into a separation tank of the organic membrane separation device and filtering through the composite polyethersulfone membrane to obtain refined Xanthoceras sorbifolium Bunge oil; weighing and adding the refined Xanthoceras sorbifolium Bunge oil into a NaOH-ethanol mixed solution, heating to obtain saponified oil, adding an equal volume of deionized water into the saponified oil for dilution, and extracting with n-hexane to obtain a mixed fatty acid;
wherein a method for preparing the two-dimensional molybdenum disulfide powder comprises the following steps: S2.1.1: adding lithium fluoride into 3 mol/L of hydrochloric acid at a solid-to-liquid ratio of 1 g:(400-500) mL, magnetically stirring for 25 min, then adding molybdenum disulfide with the same mass as the lithium fluoride, heating to 45-50° C., magnetically stirring for 5-6 h, and centrifuging and taking a precipitate to obtain a solid precipitate; and
S2.1.2: washing the solid precipitate with a LiCl solution for 5-6 times, then adding deionized water at a solid-to-liquid ratio of 1 g:(250-300) mL, subjecting to ultrasonic treatment for 30-50 min, centrifuging, taking the supernatant, and freeze-drying the supernatant at minus 20-minus 15° C. for 48-52 h to obtain two-dimensional molybdenum disulfide powder;
wherein a method for preparing the composite nanomaterial comprises the following steps:
S2.1.3: taking and adding the two-dimensional molybdenum disulfide powder prepared in the step S2.1.2 into deionized water at a solid-to-liquid ratio of 3 g:(100-200) mL, subjecting to ultrasonic treatment for 20-30 min, and magnetically stirring for 40-45 min to obtain a molybdenum disulfide dispersion; and
S2.1.4: then adding a phytic acid solution at 15-20% of a total volume of the system, subjecting to ultrasonic treatment for 30-35 min to obtain a dispersion, heating the dispersion to 180-185° C., reacting for 12-13 h, then allowing to stand and cool to room temperature of 24-26° C., centrifuging a cooled product, removing the supernatant, and freeze-drying at 0° C. for 24-25 h to obtain a composite nanomaterial;
wherein a method for preparing the composite polyethersulfone membrane comprises the following steps:
S2.1.5: taking and adding 5-10 parts by mass of piperazine into 100-150 parts by mass of deionized water, then adding 10-20 parts by mass of the composite nanomaterial prepared in the step S2.1.4, and stirring for 20-30 min to obtain a suspension;
S2.1.6: adding the suspension dropwise onto a surface of a polyethersulfone-based membrane to fully infiltrate the surface, and oven-drying under an environment of 60-65° C. after 3-5 min to obtain a primarily-treated polyethersulfone-based membrane; and
S2.1.7: adding an TMC organic phase solution dropwise onto a surface of the primarily-treated polyethersulfone-based membrane to fully infiltrate the surface, then allowing to stand for 30-50 s, and thermally crosslinking in an oven at 90-100° C. for 30-40 s to obtain a composite polyethersulfone membrane; and
S3: preparation of a nervonic acid from the mixed fatty acid
mixing the mixed fatty acid with an ethanol solution, freezing to crystallize, then performing suction filtration with a vacuum circulating water pump, and drying a filter cake to obtain a nervonic acid.

2. The process for separating and extracting a nervonic acid from Xanthoceras sorbifolium Bunge according to claim 1, wherein the preparation of Xanthoceras sorbifolium Bunge oil by extraction from a Xanthoceras sorbifolium Bunge seed kernel in the step S1 comprises the following steps:
S1.1: crushing and then sieving a dried Xanthoceras sorbifolium Bunge seed through an 80-mesh sieve, putting into an extraction kettle, sealing, vacuuming to a gauge pressure of −0.6 MPa, and then introducing tetrafluoroethane as an extraction solvent from a solvent tank into the extraction kettle until a liquid level of the extraction solvent submerges the Xanthoceras sorbifolium Bunge seed raw material as observed through sight glass;
S1.2: heating to 40-50° C. under a stirring state, and always maintaining a pressure of the extraction kettle at a gauge pressure of 1.0 MPa to fully dissolve oil in the Xanthoceras sorbifolium Bunge seed, wherein an extraction time is 50-60 min; after extraction, introducing an extract liquor into a separation tank with a pressurization pump, evaporating by heating to remove the extraction solvent, and recovering the evaporated extraction solvent vapor into a solvent tank for reuse by a system compressor through a condenser, wherein the product oil is in the separation tank and is released from the separation tank; and
S1.3: then centrifugally separating the product oil at a rotation speed of 8,000-8,200 r/min for 5-10 min, filtering, and drying at 105-110° C. for 3-3.5 h to obtain Xanthoceras sorbifolium Bunge oil.

3. The process for separating and extracting a nervonic acid from Xanthoceras sorbifolium Bunge according to claim 2, wherein the refining of the Xanthoceras sorbifolium Bunge oil by filtration through a composite polyethersulfone membrane and preparation of a mixed fatty acid in the step S2 comprises the following steps:
S2.1: replacing the organic membrane in the organic membrane separation device with the composite polyethersulfone membrane, wherein the composite polyethersulfone membrane is a polyethersulfone membrane loaded with a composite nanomaterial, and the composite nanomaterial is prepared from two-dimensional molybdenum disulfide powder as a raw material, adding the Xanthoceras sorbifolium Bunge oil into the separation tank of the organic membrane separation device, starting the pump to a rotation speed of 40 Hz, adjusting a pressure regulating valve of a feed liquid return pipe to set a pressure to 1.9 MPa, and filtering the Xanthoceras sorbifolium Bunge oil through the composite polyethersulfone membrane to obtain refined Xanthoceras sorbifolium Bunge oil;
S2.2: weighing 10-12 parts by mass of the refined Xanthoceras sorbifolium Bunge oil, adding 50-60 parts by mass of a NaOH-ethanol mixed solution, maintaining the temperature at 65-70° C. through an oil bath pot, setting a heating time to 1.5-2 h, and after end of the heating, cooling to room temperature of 24-26° C. to obtain saponified oil; and
S2.3: adding an equal volume of deionized water into the saponified oil for dilution, then adding 3 mol/L of hydrochloric acid to adjust a pH value of the system to 6-6.5, extracting with n-hexane, repeating the operation twice, collecting the n-hexane solutions from the two extractions, placing the n-hexane solutions into a rotary evaporator for concentration, and spinning the n-hexane to dryness to obtain a mixed fatty acid.

4. The process for separating and extracting a nervonic acid from Xanthoceras sorbifolium Bunge according to claim 3, wherein the preparation of a nervonic acid from the mixed fatty acid in the step S3 comprises the following steps:
S3.1: mixing the mixed fatty acid with an ethanol solution of a concentration of 90% at a solid-to-liquid ratio of 1 g:(3-4) mL, and placing in an environment of −20° C. for freezing and crystallizing for 3-4 h; and
S3.2: then performing suction filtration with a vacuum circulating water pump, ending the suction filtration when a filter cake does not contain the ethanol solution, and drying the filter cake at 20-25° C. for 24-30 h to obtain a nervonic acid.

5. The process for separating and extracting a nervonic acid from Xanthoceras sorbifolium Bunge according to claim 1, wherein a concentration of the phytic acid solution in the step S2.1.4 is 0.2-0.8 wt %.

6. The process for separating and extracting a nervonic acid from Xanthoceras sorbifolium Bunge according to claim 1, wherein the TMC organic phase solution in the step S2.1.7 is specifically a solution with a mass fraction of 2-3% formulated by dissolving trimesoyl chloride in n-hexane.

7. The process for separating and extracting a nervonic acid from Xanthoceras sorbifolium Bunge according to claim 3, wherein a volume ratio of NaOH to ethanol in the NaOH-ethanol mixed solution in the step S2.2 is 2:(3-4).
